(19) United States
(12) Patent Application Publication
(10) Pub. No.: US 2022/0076783 A1
(43) Pub. Date: Mar. 10, 2022

(54) Methods and Systems for the Precise Identification of Immunogenic Tumor Neoantigens

(71) Applicant: Merck Sharp & Dohme Corp., Rahway, NJ (US)

(72) Inventors: Razvan Cristescu, Newton, MA (US); Xinwei Sher, Wellesley, MA (US); Sybil Williams, Wayland, MA (US); Weilong Zhao, Burlingame, CA (US)

(21) Appl. No.: 17/416,121

(22) PCT Filed: Dec. 19, 2019
(86) PCT No.: PCT/US19/67460

§ 371 Date: Jun. 18, 2021

Related U.S. Application Data
(60) Provisional application No. 62/783,097, filed on Dec. 20, 2018.

(51) Int. Cl. G16B 20/30; G16B 40/10; G06N 3/08; G06N 3/04

(52) U.S. Cl. CPC G16B 20/30; G06N 3/0454; G06N 3/08; G16B 40/10

(57) ABSTRACT

An immunogenic neoantigen peptide can be identified by receiving data characterizing a neoantigen peptide from a subject. Thereafter, a naturally processed (NP) antigen predictor (NP-predictor) score can be generated using a machine learning model trained using data derived from mass spectrometry of isolated peptides eluted from at least one major histocompatibility complex (MHC) molecule. A T-epitope predictor score can be generated independently using a second machine learning model trained using experimentally characterized peptides recognized by T-cells. Additionally, a MHC binding score can be generated using a third machine learning model. The scores generated by the machine learning models can be incorporated into a composite score for each neoantigen peptide based on each of the NP-predictor score, the T-epitope predictor score, and the MHC binding score, wherein the composite score identifies one or more immunogenic neoantigen peptides.

### RELATED APPLICATION

This application claims priority to U.S. Provisional Application No. 62/783,097, filed Dec. 20, 2018, the disclosure of which is hereby incorporated by reference in its entirety.

### TECHNICAL FIELD

The subject matter described herein relates to the precise identification of immunogenic tumor neoantigens and more generally to the use of machine learning for analyzing antigens with respect to immune response, antigen presentation, and antigen processing. Specifically, provided herein are techniques for the precise identification of immunogenic tumor neoantigens as targets for a personalized cancer vaccine and for the discovery of immunotherapy or cancer biomarkers.

### BACKGROUND

Cancer is among the leading causes of death worldwide. In 2012, there were 14.1 million new cases and 8.2 million cancer-related deaths worldwide. By 2030, the global burden is expected to grow to 21.7 million new cancer cases and 13 million cancer deaths simply due to the growth and aging of the population. The future burden will probably be even larger because of the adoption of western lifestyles, such as smoking, poor diet, physical inactivity, and fewer childbirths, in economically developing countries. Existing therapeutic options often fail to take into account an individual's unique cancer makeup, which can lead to unnecessary side effects and ineffective treatment outcomes.

Cancer cells contain an accumulation of genetic alterations. Somatic mutations (e.g., non-inherited mutations, etc.) originating from the cancer cells can generate cancer-specific neoantigens that are capable of being recognized by an individual's immune system as being foreign. The successful recognition of a neoantigen can trigger an immune response that attacks and kills the cancer. For example, a neoantigen on a cancer cell can be recognized by a T-cell as being foreign, and cytotoxic T-cells can specifically target and induce cell death in the cancer cell containing the neoantigen, but not in normal cells. As a result, neoantigens represent a promising target for an anti-cancer vaccine. However, every cancer has its own unique composition of mutations, with only a small fraction shared between patients. Thus, a personalized cancer vaccine that accounts for the unique mutational landscape of an individual's cancer is necessary.

### SUMMARY

The current subject matter is directed to the identification of an immunogenic neoantigen peptide using machine learning algorithms. In a first aspect, an immunogenic neoantigen peptide is identified by receiving data derived from a subject that characterizes a neoantigen peptide from such subject. A first machine learning model is used to generate a naturally processed (NP) antigen predictor (NP-predictor) score for each neoantigen peptide. The first machine learning model can be trained using data derived from mass spectrometry of isolated peptides eluted from at least one major histocompatibility complex (MHC) molecule. In addition, a second machine learning model is used to generate a T-epitope predictor score for each neoantigen peptide. The second machine learning model can be trained using experimentally characterized peptides recognized by T-cells. A third machine learning model is used to generate a score predicting affinity of the neoantigen peptide for binding an MHC molecule (MHC binding score) for each neoantigen peptide. A composite score is then generated based on each of the NP-predictor score, the T-epitope predictor score, and the MHC binding score. The composite score can identify one or more immunogenic neoantigen peptides.

The neoantigen peptide can be identified by obtaining a sample from a subject. The sample can be derived from a cancer cell. The nucleotide sequence of one or more nucleic acids derived from the sample can be determined, and the nucleotide sequence can be translated into an encoded peptide sequence. The neoantigen peptide can be identified such that the neoantigen peptide includes a peptide sequence with at least one amino acid that differs from a corresponding wild-type peptide sequence.

Obtaining the nucleotide sequence can be performed using exome, transcriptome, and/or whole genome sequencing. In addition, the abundance of the neoantigen peptide can be determined using exome, transcriptome, and/or whole genome sequencing.

The first machine learning model can take various forms including a neural network. The neural network can be a convolutional neural network, a recurrent neural network, and/or a deep learning neural network.

The second machine learning model can take various forms for example, it can be one or more of: a support vector machine, a Bayesian classifier, a random forest model, a logistic regression model, a boosting classifier model, and/or a neural network.

The MHC molecule can be a class I or a class II. The second machine learning model can be independently selected for a specific MHC Class I human leukocyte antigen (HLA) glycoprotein, MHC Class II human HLA, or a mouse H2 glycoprotein. The HLA glycoprotein can be a HLA subtype selected from the group consisting of HLA-A, HLA-B, HLA-C, HLA-DRB, HLA-DQA, HLA-DQB, HLA-DPA, HLP-DPB, H2-Db, and H2-Kb. The HLA subtype can be a specific HLA allele to four digits.

The experimentally characterized peptides recognized by T-cells can be experimentally characterized by a human T-cell assay. The T-cell assay can measure cytokine release, cytotoxicity, or qualitative T-cell binding to an antigen presenting cell (APC). The cytokine measured for a T-cell assay can be selected from the group consisting of interferon gamma (IFNγ), tumor necrosis factor alpha (TNFα), interleukin-2 (IL-2), interleukin-4 (IL-4), interleukin-5 (IL-5), interleukin-6 (IL-6), interleukin-8 (IL-8), interleukin-10 (IL-10), interleukin-17 (IL-17), interleukin-21 (IL-21), interleukin-22 (IL-22), granzyme A, and granzyme B. In some variations, the qualitative T-cell binding can be determined by MHC multimer staining. In some variation, the T-cell assay can be performed ex vivo or in vitro. The experimentally characterized peptides recognized by T-cells can be selected from the Immune Epitope Database (IEDB). Some variations further include subjecting the experimentally characterized peptides recognized by T-cells to dimensionality reduction. The dimensionality reduction can include principal component analysis (PCA), singular value decomposition (SVD), or non-negative matrix factorization (NMF). In some variations, the T-cell assay readout can be positive or negative.

Some variations further include separating a neoantigen peptide longer than nine amino acids into segments of nine amino acids with a same sequence order. In some variations, the peptide is a 9-mer antigen peptide. The nine 9-mer neoantigens can serve as the input for the third machine learning model.

The third machine learning model can take various forms including a neural network. In some variations, the neural network source of the third machine learning model can be NetMHC.

Some variations further include generating, for each neoantigen peptide, an ImmunoGenScore determined by a summation of the NP-predictor score, and the T-epitope predictor score. In some variations, the composite score is generated, for each neoantigen peptide, by a summation of the NP-predictor score, the T-epitope predictor score, and the MHC binding score. In other variations, the composite score is generated for each neoantigen peptide by an orthogonal scoring matrix. The orthogonal scoring matrix can include generating, for each neoantigen peptide, an orthogonal rank for the ImmunoGenScore. The orthogonal scoring matrix can also include generating, for each neoantigen peptide, an orthogonal rank for the MHC binding score. In some variations, neoantigen peptides with a high orthogonal rank for the ImmunoGenScore, and a high orthogonal rank for the MHC binding score can be identified. Neoantigen peptides present in either or both of the highest orthogonal rank for the ImmunoGenScore and the highest orthogonal rank for the MHC binding score can be selected as a predictive immunogenic neoantigen peptide.

In some variations, a high orthogonal rank for the ImmunoGenScore can be a neoantigen peptide in the top 50% of neoantigen peptides. In some variations, a high orthogonal rank for the MHC binding score can be a neoantigen peptide in the top 10% of neoantigen peptides.

Some variations further include prioritizing neoantigen peptides with the highest expression. In some variations, the expression is determined by RNA-seq.

The immunogenic neoantigen peptide can be a target for an anti-cancer vaccine. In some variations, a higher composite score indicates the immunogenic neoantigen peptide is an optimal target for an anti-cancer vaccine. In some variations, the composite score is a personalized cancer vaccine (PCV) score.

Non-transitory computer program products (i.e., physically embodied computer program products) are also described that store instructions, which when executed by one or more data processors of one or more computing systems, cause at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including but not limited to a connection over a network (e.g., the Internet, a wireless wide area network, a local area network, a wide area network, a wired network, or the like), via a direct connection between one or more of the multiple computing systems, etc.

The subject matter described herein provides many technical advantages. In particular, the current subject matter provides a precise and accurate prediction of immunogenic neoantigen peptides that are expected to undergo antigen processing, MHC-presentation, and T-cell interaction. Furthermore, the current subject matter has significant application potential in the design of personalized cancer vaccines and the discovery of immunotherapy biomarkers.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is a first process flow diagram illustrating prediction of an immunogenic neoantigen peptide.

FIG. 2 is a second process flow diagram illustrating prediction an immunogenic neoantigen peptide.

FIG. 3 illustrates an exemplary system for predicting immunogenic tumor neoantigens.

FIG. 4 illustrates construction of a neural network for generating a naturally processed (NP)-predictor score.

FIG. 5 illustrates that NP-predictor demonstrates higher specificity, and combination of specificity with sensitivity (F1 score) on training data free MHC-eluted, NP peptides detected from HLA mono-allelic cell lines.

FIG. 6 illustrates that the NP+Tepi (ImmunoGenScore) outperforms NP-predictor and NetMHC on human melanoma-derived immunogenic neoantigens.

FIG. 7 illustrates that the ImmunoGenScore outperforms NetMHCpan3, NETMHC3, and NETMHC4.

FIG. 8 illustrates that the ImmunoGenScore performs better than the NetMHC alone for predicting neoantigens that elicit cytotoxic T-cell (CD8+) reactivity.

FIG. 9 illustrates the orthogonal scoring system for selecting immunogenic neoantigens. MHC-binding affinity IC50 value is normalized to [0, 1] scale. ImmunoGenScore is computed as the addition of NP- and T-epitope-predictor scores. Double positive region (shaded) indicates neoantigens with strong immunogenic potency.

### DETAILED DESCRIPTION

Provided herein are variations for the current subject matter related to a precise identification of immunogenic neoantigens using machine learning. Neoantigens are a class of HLA-bound peptides that arise from cancer-specific mutations. Specifically, as provided herein a “neoantigen” is intended to mean a unique, new antigen to a specific cancer, tumor, or cell thereof, which arises as a consequence of the accumulation of random mutations from aberrant DNA replication and/or repair in the cancer, tumor, or cell thereof. Effective anti-cancer immunity in humans has been associated with the presence of T-cells directed at cancer neoantigens. Thus, identification of immunogenic neoantigens represent a promising target for anti-cancer vaccines.

Advances in sequencing methods, and in silico analysis of tumor-specific mutations has enabled the identification of neoantigens. However, not all neoantigens are capable of eliciting an effective immune response. Therefore, the current subject matter can, in some variations, use multiple, independent machine learning models to evaluate and identify optimal immunogenic neoantigen peptides. This arrangement is achieved by training the machine learning models using, for example, data from high-quality mass spectrometry (MS) of isolated peptides eluted from a major histocompatibility complex (MHC) molecule, and experimentally characterized peptides recognized by T-cells, as well as incorporating the prediction output for a peptide's MHC binding affinity. The integration of the output from the machine learning models into a single composite score can enable the precise identification of personalized cancer vaccine candidates.

Genome instability and mutations are a hallmark of cancer. When the mutations are not inherited and occur at some time after conception, the mutations are termed somatic mutations. The somatic mutations can be present in a specific population of cells. For example, when the mutation leads to a malignant growth the somatic mutation can be unique to the cancer cells. When the somatic mutations occur in the coding regions of the genome, the mutations have the potential to generate neoantigens.

The neoantigens are important from an immunology standpoint, because the neoantigens are antigens to which the immune system has not been previously exposed. As a result, neoantigens can represent a vulnerability for cancer cells if they become recognized by the immune system as foreign. The successful recognition of a neoantigen on a cancer cell can trigger an immune response to specifically target and destroy the cancer cells. Therefore, neoantigens are considered important targets for cancer immunotherapy because of their immunogenicity and lack of expression in normal tissues.

Neoantigens can be identified by determining the nucleotide sequence of a cancer cell and comparing it to a reference sequence. In some variations, the reference sequence can be a normal cell from the same subject. For example, a biopsy can be collected from a subject and sequencing can be performed on the cancer cells and the normal cells adjacent to the cancer. In other variations, the reference sequence can be from a publicly available sequence of a healthy subject or a consensus sequence of multiple healthy subjects. For example, the reference sequence can be derived by determining the nucleotide sequence in two or more healthy subjects and generating a consensus sequence based on perfect homology between the subjects.

DNA and/or RNA can be prepared using, for example, nucleic acid purification methods. Subsequently, the nucleic acids can be sequenced using, for example, Sanger sequencing, Next-generation sequencing, or related methods capable of identifying the precise sequence of nucleotides in the DNA and/or RNA. For example, sequencing can be performed by whole genome sequencing (WGS) of a subject's DNA, or specific subsets of the genome can be sequenced, such as whole exon sequencing (WES). In addition, gene expression analysis by RNA sequencing (RNA-seq), that is transcriptome sequencing, or microarrays can be used to predict candidate neoantigens derived from the somatic mutations detected by WES.

Differences between mutated peptide sequence in cancer cells and the wild-type peptide sequence can identify potential neoantigen peptides. For example, the nucleotide sequence of the exonic DNA or the messenger RNA from a cancer cell can be translated into an encoded peptide sequence. Exemplary wild-type peptide sequences can be obtained by sequencing the nucleotide sequence of the exonic DNA or the messenger RNA from a normal tissue and translating the nucleotide sequence into an encoded peptide sequence. Additional wild-type peptide sequences can be used as a reference, and need to be from a paired sample from a normal tissue. For example, a consensus wild-type peptide sequence obtained from patient's blood or two or more healthy subjects can serve as the wild-type peptide sequence. Peptide sequences originating from the cancer cells that contain at least one amino acid that differs from a corresponding wild-type peptide sequence can be considered a neoantigen peptide.

The sequencing can be performed on various samples collected from a subject having or suspected of having cancer. Exemplary samples include a tumor biopsy, a benign sample, or a blood sample with circulating tumor DNA. The sequencing can be performed on a single cell, or a pool of cells. In silico tools can be used to identify mutations and the encoding neoantigen peptides.

The abundance of a neoantigen peptide can also influence the efficacy of a neoantigen peptide as a target for an anti-cancer vaccine. For example, a neoantigen peptide that is expressed at low levels on a cancer cell can be unlikely to be encountered by a T-cell, even if the neoantigen has been presented to effector T-cells and memory T-cells have been formed. Conversely, a neoantigen peptide that is expressed at relatively high levels can have a higher probability of encountering T-cells surveillance. The abundance of the neoantigen peptide can be determined by sequencing methods, such as those described above. Abundance can be determined by gene expression, as well as allelic expression. Therefore, in some variations, the current subject matter provided herein further includes determining the abundance of the neoantigen peptide using exome, transcriptome, or whole genome sequencing.

Using the teachings provided herein, the abundance of neoantigen peptides can be determined. In some variations, the neoantigen peptides with the highest expression can be prioritized as being the best candidates for an anticancer vaccine. In specific variations, the expression can be calculated from sequencing RNA. In other variations, the expression can be determined by sequencing the expression of specific alleles that contain somatic mutations.

Provided herein “cancer” includes, but is not limited to, solid cancer and blood borne cancer. The term “cancer” refers to disease of tissues or organs, including but not limited to, cancers of the bladder, bone, blood, brain, breast, cervix, chest, colon, endometrium, esophagus, eye, head, kidney, liver, lymph nodes, lung, mouth, neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastase, glioblastoma multiforms, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, colorectal cancer, including stage 3 and stage 4 colorectal cancer, unresectable colorectal carcinoma, metastatic hepatocellular carcinoma, Kaposi's sarcoma, karotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unrescectable hepatocellular carcinoma, Waldenstrom's macroglobulinemia, smoldering myeloma, indolent myeloma, fallopian tube cancer, androgen independent prostate cancer, androgen dependent stage IV non metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

Computational approaches can be employed to facilitate identification of immunogenic tumor neoantigens using machine learning algorithms. As disclosed herein, a computational immunogenic neoantigen prediction pipeline can be employed that combines machine learning predictors trained on (i) Mass Spectrometry (MS) data of peptides eluted from a MHC molecule, (ii) T-cell activation/interaction assay output data, and/or (iii) prediction of MHC-binding affinity.

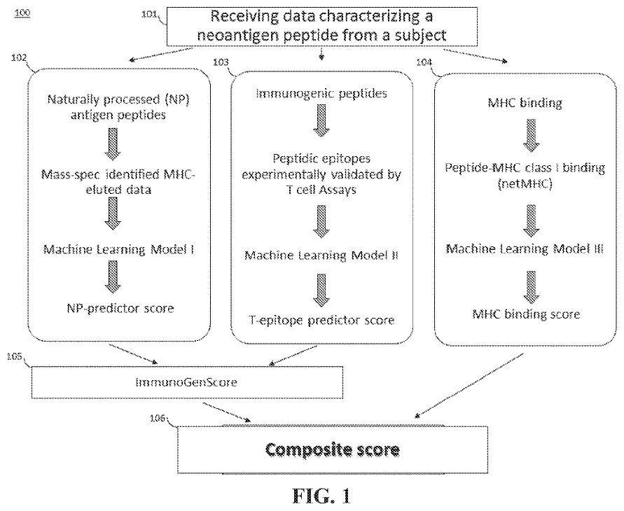

FIG. 1 is a process flow diagram 100 illustrating the identification of an immunogenic peptide variation. Initially, at 101, data is received that characterizes a neoantigen peptide from a subject as input to one or more data processors forming part of at least one computing device. In some variations, the neoantigen peptides can be between 9 to 23 amino acids in length. In specific variations, the neoantigen peptides that serve as input to a machine learning algorithm are exactly nine amino acids in length. Consequently, in some variations, the input to a machine learning algorithm can comprise all of the various sequences of nine amino acids that originate from a larger peptide with the same amino acid sequence order. For example, if a neoantigen peptide is 17 amino acids in length, then the input for that specific neoantigen peptide can comprise each of the nine different possible combinations of nine amino acids that can be generated from the same sequence order of amino acids.

In some variations, the neoantigen peptide is used, at 102, to generate a naturally processed (NP) antigen predictor score (NP-predictor) score. This can be achieved using a first machine learning model that is trained using data derived from mass spectrometry (MS) of isolated peptides eluted from at least one MHC molecule. High-throughput, high-quality MS experiments have identified a number of naturally processed (NP) antigen peptides from various pathogens, cancer cell lines and human tumor samples. These peptides represent bona fide antigens that are processed by antigen presenting cells and presented on MHC molecules. Furthermore, a large fraction of these peptides is not accurately predicted using MHC-binding prediction algorithms alone.

Identification of the NP peptides eluted from MHC can be performed by immunoprecipitation of MHC molecules followed by peptide elution, purification, and analysis by liquid chromatography-MS/MS. The elution data can include MHC class I and MHC class II data. Further, the data can be generated from human and non-human samples, such as mouse. Peptides identified from elution of MHC molecules represent validated MHC-presenting epitopes that can provide an accurate prediction of peptides that are likely to elicit T-cell receptor interactions. As such, the naturally processed peptides can complement or enhance output information generation from the prediction of MHC-binding affinity.

The data that is used for training the machine learning algorithm for a NP predictor score can be selected and curated from experimental evidence of peptides eluted from MHC molecules. For example, the data can be collected from publicly available experimental results and include the peptide sequence, associated HLA allele type, source protein ID, MS abundance percentage, and predicted or experimentally measured HLA-binding affinity.

The data used for determining the NP predictor score need not be from mono-allelic cells. The data can also be determined from multi-allelic cells. For example, for multi-allelic cells the HLA allele with the strongest binding to the eluted peptide can be designated as the associated allele. Alternatively, a MHC binding affinity prediction model can be used for multi-allelic cells without experimental data on HLA binding affinity. For example, a peptide can be subjected to an MHC binding affinity prediction model, and among the eluted HLA alleles the one that is predicted to have the strongest binding to the eluted peptide can be designated as the associated allele.

In some variations, the MS abundance percentage (0-100%) can be incorporated into the machine learning algorithm. For example, a peptide with an abundance of 94.2% can be incorporated as 0.942. Alternatively, a peptide that does not have experimental data on peptide abundance can be treated as 100% and be assigned a value of 1.0. For example, a peptide without abundance data, but detected from the MHC eluted samples, can be set at 1.0. Therefore, any peptide detected from the MHC eluted samples can be given an MS abundance value greater than 0 and less than or equal to 1.0.

The machine learning algorithm for generating an NP-predictor score can also be trained using negative labels. Exemplary negative labels can be generated from random sequences of nine amino acids collected from the same or similar set of source genes used to map the positive human proteome database. For example, the MS-positive peptides and the random sequences can be generated using the human proteome database. A local alignment with the positive MS-identified peptides can be performed to exclude any sequences where more than four amino acid residues were aligned between the positive and the negative peptides. The number of negative decoys can be 1,000 peptides, 5,000 peptides, 10,000 peptides, 50,000 peptides, 100,000 peptides, or 500,000 peptides or more peptides that are not present in the MHC-eluted peptide data as decoys. Such negative labels can be given a label of 0.0 that corresponds with the abundance score of the positive peptides described above.

Various machine learning algorithms can be used to analyze the neoantigen input against the mass-spec identified MHC-eluted data to generate a NP-predictor score (step 102). In some variations, the first machine learning model is a neural network (NN) that generates a NP-predictor score. In specific variations, the neural network is a convolutional neural network, a recurrent neural network, or a deep learning neural network. It is understood that the first learning model can also generate a NP-predictor score using other machine learning algorithms and that the neural network algorithm is intended to be exemplary. For example, in some variations, the first machine learning model is a random forest, logistic regression, or an unsupervised clustering model.

An exemplary first machine learning model using a random forest or a logistic regression model can utilize training data input where the NP peptide sequences and the output is a class label of positive vs. negative. Alternatively, an exemplary first machine learning model can use an unsupervised clustering model that includes training data input from NP peptide sequences identified from a list of, for example, specific human HLA types such that the model assigns each sequence to a unique HLA type. The predictor therefore predicts the possibility of one particular sequence being eluted/naturally presented by that specific HLA type.

In other variations of neural network models, the model can also be a deep neural network (DNN) with multiple locally and fully connected hidden layers, or a high-order neural network (HONN). For DNN, a Restricted Boltzmann Machine (RBM) can be used to pre-train the neural nodes of input and connecting layers. For HONN, a mean-covariance RBM can be used to pre-train the neural nodes of input and connecting layers.

Transformation of a peptide sequence into a numerical value can be performed by representing each amino acid by a 1*1 vector ranging from 1 and 20, where the numerical value corresponds with one of the twenty different amino acids. For example, valine can be assigned “1”, alanine “2”, arginine “3”, glutamine “4”, lysine “5”, and so on for all twenty amino acids. It is understood that the numbers are assigned arbitrarily and any number can be assigned to any amino acid, as long as the number assigned for a given amino acid is consistent for all peptides. Subsequently, the 1*1 vector can be transformed into a 1*16/32 vector by the automatic embedding layer of the machine learning algorithm, such as a neural network.

The prediction can be further classified by modifying the activation functions of the embedding-to-dense and dense-to-output layers. For variations of activation functions, such as ReLU or sigmoid, the predictor gives different signal mapping of non-linear relationship between layers, therefore adjusting the ability of the predictor to learn the different relationships. In some variations, a rectified linear unit (ReLU) can be used for the activation functions. In some variation, a sigmoid or a hyperbolic tangent (tan h) can be used for the activation functions.

Prediction can be further trained for each HLA allele using the positive peptides that correspond only to the specific HLA allele. Specificity of the HLA allele can be up to four digits (e.g. HLA-A*01:01). Further, the negative peptides can be selected from the pool of peptides described previously. Consequently, each HLA allele can have a range of positive to negative peptides, depending on the number of positive peptides that are detected for each HLA type. For example, the positive to native ration can be about 1:30, 1:40: 1:50, 1:60, 1:70, 1:80, 1:90, or about 1:100, and any integer in-between.

The output of the NP prediction algorithm can be a floating number ranging from 0 to 1. For example, a neoantigen peptide used as an input can be subjected to the NP predictor and given a score of 0 if the neoantigen peptide is not a peptide detected on MHC-eluted molecules identified from MS. Alternatively, a neoantigen peptide can have a score of 1.0 for HLA-A*01:02 and 0.0 for HLA-B*51:01 if the peptide has detected specifically on the HLA-A*01:02 allele, but not on the HLA-B*51:01, and no information of the MS abundance was provided. It is understood that these are exemplary scenarios and that the NP-prediction scores will be specific to a given individual and are not universal for all subjects.

The affinity that a neoantigen peptide has for an MHC molecule is an additional factor that can influence the immunogenicity of a neoantigen peptide. Although prediction of the binding affinity alone does not accurately predict the efficacy of a peptide in being able to elicit an immune response, the binding of a peptide to an MHC molecule is an integral step in the process of a peptide eliciting an immune response. Therefore, in some variations, at 104, the neoantigen peptide is received by a machine learning model to generate a score predicting affinity of the neoantigen peptide for binding an MHC molecule (MHC binding score).

The prediction of peptide-MHC binding can be performed using machine learning algorithms. In some variations, the machine learning algorithm for predicting peptide-MHC binding is an artificial neural network. The ANN can be trained for the different MHC alleles, including HLA-A, HLA-B, HLA-C, and HLA-E. In some variations the HLA allele is specific to 4-digits, for example HLA-A*01:01. Publicly available prediction tools are available for predicting peptide-MHC binding. In some variations, the NetMHC server can be employed for prediction of peptide-MHC class I binding using artificial neural networks (ANNs).

The neoantigen peptide sequences used as input for generating a MHC-binding score can be made of any length. Most HLA molecules have a strong preference for binding sequences of nine amino acids (9-mers), so the machine learning algorithm for predicting the MHC binding score can be more accurate when the peptide input is about nine amino acids in length. Therefore, in some variations, the input for a MHC binding score is an 8-mer, 9-mer, 10-mer, or 11-mer peptide. In specific variations, the input for a MHC binding score is a 9-mer. In other variations, the input to a machine learning algorithm can comprise all of the various sequences of nine amino acids that originate from a larger peptide with the same amino acid sequence order. For example, if a neoantigen peptide was 17 amino acids in length, then the input for that specific neoantigen peptide can comprise nine different combinations of nine amino acids. The predicted binding affinity (IC50, nM) can be normalized using a [0,1] scale by log-transformation: (1−log 10(IC50)/log 10(50000.0)), where 1 indicates the strongest binding affinity.

The identification of neoantigen peptides that can be naturally processed and presented on an MHC molecule are important determinants for whether a neoantigen can engage a T-cell and exhibit an immune response. However, antigen processing and an antigen's affinity for MHC binding are not necessarily indicative of T-cell activation. For example, a peptide can be naturally processed, have strong affinity for an MHC molecule, or both, and yet exhibit little to no T-cell activation. Consequently, the identification of an immunogenic neoantigen peptide can be aided further by analyzing peptides that have a functionally validated immune response to a known peptide sequence. Therefore, in some variations, at 103, the neoantigen peptide is received by a second machine learning model to generate a T-epitope predictor score, where the machine learning model is trained using experimentally characterized peptides recognized by T-cells.

Various parameters can be used to measure a T-cell response. Exemplary experimental evidence of T-cell activation includes secretion of specific cytokines (e.g., interleukins, interferon-gamma, tumor necrosis factor alpha, and granzymes A and B), proliferation of T-cells, functional responses such as cytotoxicity, and qualitative T-cell binding to an antigen presenting cells (APC). In some variations, the machine learning model for a T-epitope predictor score is trained using data collected from T-cell assays of experimentally characterized peptides recognized by T-cells. In specific variations, the T-cell assays can measure cytokine release, cytotoxicity, or qualitative T-cell binding to an APC. Additional evidence can also be considered, and it is understood that the above experimental evidence is not intended to be inclusive of all indicators or T-cell activation.

Markers of T-cell activation can be used to determine immunogenicity of a peptide. One marker of T-cell activation is cytokine release. Many cytokines and their function of the immune response are well defined. An exemplary function of a well-defined cytokine is the ability of interleukin-2 (IL-2) to stimulate growth of T-cells. Similarly, interleukin-4 (IL-4) can stimulate growth, as well as survival, of T-cells. Tumor necrosis factor alpha (TNFα) is another well-defined cytokine secreted by T-cells that can activate macrophages and induce nitric oxide production, which is a powerful cytotoxic chemical. In some variations, the T-cell assay measure cytokine release. In specific variations, the cytokine is selected from the group of interferon gamma (IFNγ), tumor necrosis factor alpha (TNFα), interleukin-2 (IL-2), interleukin-4 (IL-4), interleukin-5 (IL-5), interleukin-6 (IL-6), interleukin-8 (IL-8), interleukin-10 (IL-10), interleukin-17 (IL-17), interleukin-21 (IL-21), interleukin-22 (IL-22), granzyme A, granzyme B, or any combination thereof.

The data for generating the experimentally characterized peptides from cytokine release can be from any assay used to measure cytokine release. For example, the T-cell assay for measuring IFNγ release can be an ELISPOT, ELISA, or similar assay that measures cytokine release. Data can be collected from other exemplary assays that measure cytokine release, and it is understood that the examples described above are intended to be exemplary. In addition, it is understood that the experimental data need not include information on all, or any, of the cytokines mentioned above, and that cytokine release is merely one type of assay that can used to characterized a neoantigen peptide that elicits a T-cell response.

Another exemplary T-cell assay from which data characterizing a neoantigen's immunogenicity can be drawn is a qualitative T-cell binding assay. In some variations, the qualitative T-cell binding assay data is multimer/tetramer qualitative binding. For example, the machine learning algorithm can analyze data from a flow cytometry assay that provides experimental evidence on cell-cell binding of a T cell epitpose:MHC:T-cell receptor (TCR) complex. Another exemplary assay includes a MHC tetramer staining to identify peptides that are recognized by a T-cell. The machine learning algorithm for generating a T-epitope predictor score can incorporate data from additional T-cell assays, including those not described previously, and the assays describe above are intended to be exemplary support of the type of experimental evidence that can be used for training the machine learning model using experimentally characterized peptides recognized by T-cells.

Yet another exemplary T-cell assay that can be used for characterizing a neoantigen's immunogenicity is a T-cell mediated cytotoxicity assay. For example, the assay can measure the directed killing of a target cell by a T-cell through the release of granules containing cytotoxic mediators or through the engagement of death receptors. In certain variations, the assay for measuring cytotoxicity is a chromium-51 release assay. Measurement of cytotoxicity can be performed by a variety of assays, and the examples provided above are understood to be merely exemplary support of the type of experimental evidence that can be used for training the machine learning model using experimentally characterized peptides recognized by T-cells.

The neoantigen peptide sequence used as input for generating the T-epitope predictor score can be of any length. For example, in some variations, the neoantigen peptide is less than or equal to 50 amino acids, less than or equal to 40 amino acids, less than or equal to 30 amino acids, less than or equal to 20 amino acids, or less than or equal to 10 amino acids. In specific variations, the neoantigen peptide is nine amino acids.

The data collected from the experimentally characterized peptides recognized by T-cells can include binary class labels. For example, a neoantigen peptide that has been experimentally validated to activate T-cells by a T-cell assay, such as any one of the assays describe previously, can be considered “positive” and be assigned a value of 1.0. An alternative example can be a neoantigen peptide that has been characterized to not elicit any activation of T-cells. Such an exemplary peptide would be assigned a value of 0.0. Any assay that measures T-cell activation can be employed, whether or not it has been described previously, and the peptide can be assigned a binary class label according to the experimentally characterized peptides.

The experimentally characterized peptides recognized by T-cells can be collected from experiments performed by the user, by experiments selected from a public database, or both. An exemplary database of experimentally characterized peptides recognized by T-cells is the Immune Epitope Database (IEDB). Therefore, in some variations, the experimentally characterized peptides recognized by T-cells are selected from IEDB. However, any database of experimentally characterized peptides can be used for the T-epitope predictor score, and it is understood that IEDB is an exemplary database. In some variations, data from a database can be combined with personal data that is not found in the public database. For example, a user can compile data from publications, generate new data, or acquire data that is not found in the database and use it for training the machine learning model. The data used to train the machine learning model for the T-epitope predictor score can be from ex vivo, in vitro, or in vivo data. In certain variations, the data is from ex vivo or in vitro restimulation experiments.

The machine learning model for generating the T-epitope predictor can also be configured to consider specific HLA and/or H2 alleles. In certain variations, the HLA can be specific to the HLA locus and be selected from the group of HLA-A, HLA-B, HLA-C, HLA-DRB, HLA-DQA, HLA-DQB, HLA-DPA, or HLA-DPB. In some variations, the HLA allele can be specific to 4-digits (e.g. HLA-A*02:01). In some variations, the H2 alleles can be specific to the mouse H2 locus and be selected from the group of H2-Db or H2-Kb.

The algorithms for the machine learning model used to generate the T-epitope predictor can be selected from the group of a support vector machine, a Bayesian classifier, a random forest model, a logistic regression model, a boosting classifier, or a neural network. Depending on the amount of training data available for certain HLA alleles, specific algorithms may be better suited for a given dataset that relates to a specific HLA allele. For example, a neural network algorithm can be ineffective for a dataset with limited training data.

The selection of a specific machine learning algorithm for a specific HLA can be performed using a set of models, such as boosting regression, random forest, or support vector machine. The models can be trained and tested on the same 3-folds cross-validation dataset. For example, the training data can be split into three subsets (e.g. A, B, and C) and the model can be trained using two of the different subsets (e.g. A and B). The remaining subset (e.g. C) can then be used test the fitness of the algorithm. The process can be repeated using different permutations of the subsets until the model with the best auROC or lowest mean absolute error can be selected for a given HLA type. Finally, the model can then be re-trained using the entire data available unique to that HLA type.

Therefore, in specific variations, the algorithm of the machine learning model for generating a T-epitope prediction score can be specific for a given HLA allele. For example, the HLA-A subtype can use the boosting classifier, the HLA-B subtype can use the Bayesian classified, and the HLA-C subtype can use the logistic regression model. In specific variations, the algorithm of the machine learning model for generating a T-epitope prediction score can be specific for a given HLA allele defined by 4-digits. For example, the HLA-A*01:08 can use the boosting classifier, the HLA-A*01:01 can use the random forest model, the HLA-A*01:01 can also use the random forest model, the HLA-B*07:02 can use the support vector machine, and so forth for all of the specific HLA alleles that have experimental evidence. The examples provided above are merely exemplary, and it is understood that the algorithm for each specific HLA allele can be any algorithm selected from the group described above. Further, an algorithm can be independently selected for each specific HLA allele. For example, selection of a boosting classifier algorithm for HLA-A*24:02 does not affect the selection of the algorithm to be used for any other specific allele. Therefore, in some variations, a machine learning algorithm for generating a T-epitope prediction score will be independently assigned to a specific HLA allele.

The best algorithm for each HLA allele can be determine by taking an average of the 3 folds cross-validation of an area under receiving operative characteristic curve (auROC). Parameters can be set for each algorithm. For example, a penalty of error term of 0.05 can be set for SVM. Similarly, a number of decision tree estimators of 10, and a maximum features to consider can be 30 for a random forest. Further, for a neural network, a 5-fold validation using 80% to 20% training data splitting to optimize the hyperparameters including number of nodes in the embedding and dense layers, and the drop-out ratio. The training data can be split into five subsets (e.g. A, B, C, D, and E), and the model can be trained five times such that one of the datasets is omitted during the training and used for testing. For example, A, B, C, and D can be used for training and E can be used for testing. Similarly, B, C, D, and E can be used for training and A can be used for testing. This training and testing permutations can be performed for each of the five different combinations. The hyperparameter set with the best combination of auROC, F1 score, and Kendall's T, averaged on the five left-one tests can be selected as the final hyperparameter for neural network.

The input sequence can be encoded with 1/0 hotshot approach. This means each amino acid was represented by a 1*20 vector, with the position of that amino acid set to 1 and the rest set to 0. For a 9mer peptide the total vector length is 180. The feature space was then reduced to a 1*12 vector by means of dimensionality reduction, including Principle Component Analysis (PCA), Singular Value Decomposition (SVD), or Non-negative Matrix Factorization (NMF).

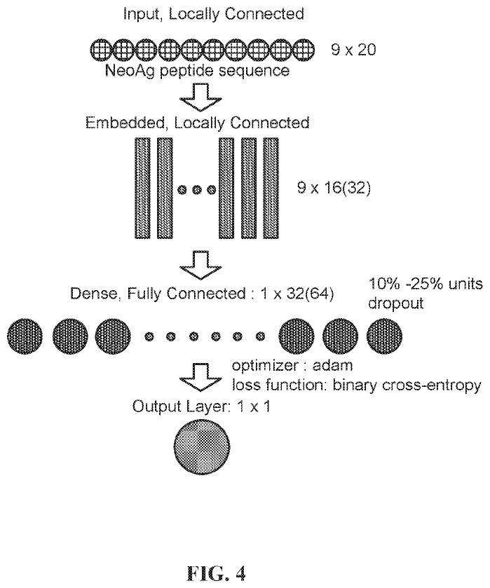

For ANN, each amino acid was first represented by a 1*1 vector between 1 and 20, and then to a 1*16/32 vector by the automatic embedding layer of ANN (FIG. 4). Upon completion of algorithm cross-validation, the allele-specific model with the best performing algorithm was saved for prediction task (Table 1 for HLA class covered).

The data collected from the experimentally characterized peptides recognized by T-cells can also be subjected to dimensionality reduction. In some variations, the dimensionality reduction includes principal component analysis (PCA), singular value decomposition (SVD), or non-negative matrix factorization (NVF).

The output generated from the NP-predictor score and the T-epitope predictor score can be combined together, at 105, to generate an ImmunoGenScore. The ImmunoGenScore incorporates the likelihood of both MHC-presentation and T-cell receptor (TCR) interaction. ImmunoGenScores can then be compiled into a ranking list of immunogenic neoantigen peptides that are ordered from highest to lowest, with the highest score being ranked first. Similarly, a separate ranking list can be generated for the neoantigen peptides according to their MHC binding score, with the highest score being ranked first. Subsequently, peptides that are the highest in both rankings can be identified as immunogenic neoantigen peptides. For example, in certain variations, the top 50% of neoantigen peptides from the ImmunoGenScore ranking list can be selected, and the top 10% of neoantigen peptides from the MHC binding score ranking list can be cross-referenced. Peptides that are common to both can, at 106, be selected and given a composite score based on their ranking in both lists. The criteria for selecting the top peptides in each list can be adjusted according to the user's preference. For example, the top 50%, 40%, 30%, 20%, 10%, 5%, 1% or any number in-between can be selected from one or both lists. It is understood that more stringent criteria will yield fewer neoantigen peptides being identified. However, it is also understood that the more stringent criteria can more accurately predict neoantigen peptides that are immunogenic. Alternatively, less stringent conditions will yield more neoantigen peptides, and can include more peptides that are not immunogenic.

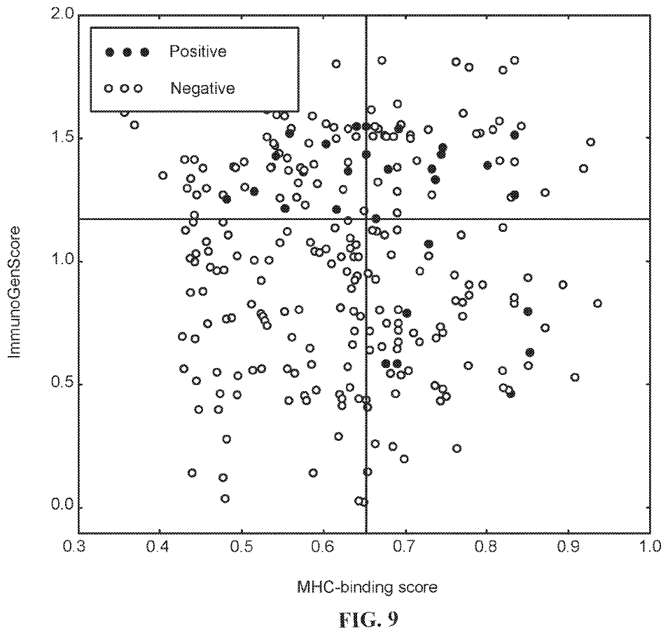

In certain variations the ranking lists for the ImmunoGenScores and the neoantigen peptides according to their MHC binding score can be organized numerically from low to high as independent orthogonal rankings. The orthogonal rankings can then be combined into a matrix to form an orthogonal scoring matrix that is able to generate a composite score. As provided herein, an orthogonal scoring matrix is intended to mean an independent ranking of two or more variables into separate orthogonal rankings that are then compared in parallel in order to provide a candidate peptide with a composite score that factors in the ranking of each variable. An exemplary orthogonal scoring matrix is an XY matrix, such as illustrated in FIG. 9, where the one variable, such as for example a “MHC-binding score,” is on the X-axis and another variable, such as for example an “ImmunoGenScore,” is on the Y-Axis. This type of orthogonal scoring matrix can identify candidate peptides that have both high ImmunoGenScores and high MHC-binding scores by their location in the top right quadrant. However, it is understood that other types of orthogonal scoring matrices can be generated and that the exemplary orthogonal scoring matrix described above is not intended to be limiting.

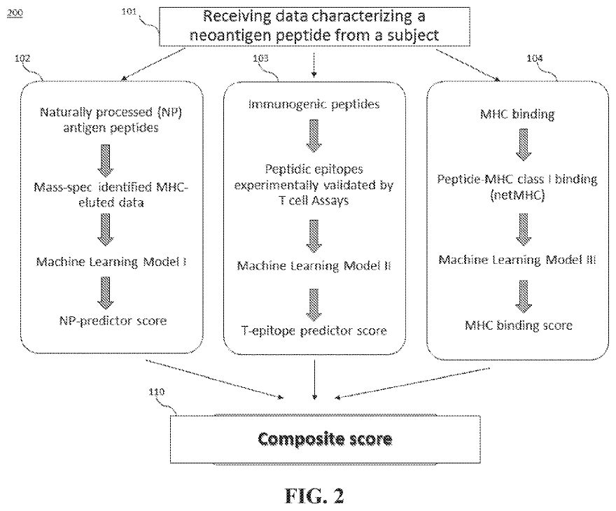

FIG. 2 illustrates steps in an alternative system where the three separate machine learning models can be directly summed together to generate a composite score at 110. For example, a peptide with a NP-predictor score of 0.5, a T-epitope predictor score of 1.0, and an MHC-binding score of 0.5 can have a composite score of 2.0. An alternative exemplary composite score can be 1.0 for a peptide with a NP-predictor score of 0.25, a T-epitope predictor score of 0, and an MHC-binding score of 0.75. Such a composite score would be on a scale of 0.0 to 3.0. A composite score can be generated for each of the neoantigen peptides using the machine learning models provided herein. Peptides with the highest score will be predicted to be immunogenic, whereas those with a lower score will be predicted to be non-immunogenic.

It is understood that the combined score can be used to rank the immunogenicity. It is also understood that a higher composite score predicts a neoantigen peptide to be immunogenic, and an optimal target for an anti-cancer vaccine. Thus, in some variations, the composite score is a personalized cancer vaccine score.

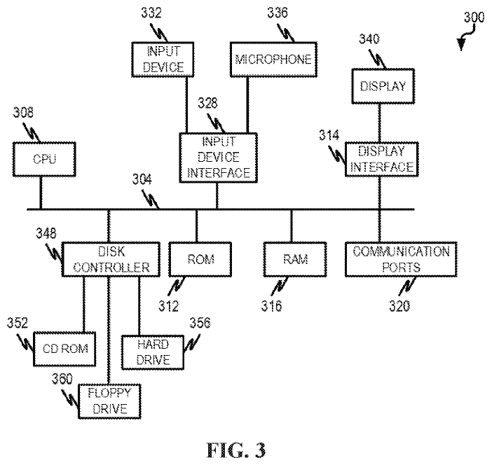

FIG. 3 is a diagram 300 illustrating a sample computing device architecture for implementing various aspects described herein. A bus 304 can serve as the information highway interconnecting the other illustrated components of the hardware. A processing system 308 labeled CPU (central processing unit) (e.g., one or more computer processors/data processors at a given computer or at multiple computers), can perform calculations and logic operations required to execute a program. A non-transitory processor-readable storage medium, such as read only memory (ROM) 312 and random access memory (RAM) 316, can be in communication with the processing system 308 and can include one or more programming instructions for the operations specified here. Optionally, program instructions can be stored on a non-transitory computer-readable storage medium such as a magnetic disk, optical disk, recordable memory device, flash memory, or other physical storage medium.

In one example, a disk controller 348 can interface one or more optional disk drives to the system bus 304. These disk drives can be external or internal floppy disk drives such as 360, external or internal CD-ROM, CD-R, CD-RW or DVD, or solid state drives such as 352, or external or internal hard drives 356. As indicated previously, these various disk drives 352, 356, 360 and disk controllers are optional devices. The system bus 304 can also include at least one communication port 320 to allow for communication with external devices either physically connected to the computing system or available externally through a wired or wireless network. In some cases, the communication port 320 includes or otherwise comprises a network interface.

To provide for interaction with a user, the subject matter described herein can be implemented on a computing device having a display device 340 (e.g., a CRT (cathode ray tube) or LCD (liquid crystal display) monitor) for displaying information obtained from the bus 304 to the user and an input device 332 such as keyboard and/or a pointing device (e.g., a mouse or a trackball) and/or a touchscreen by which the user can provide input to the computer. Other kinds of input devices 332 can be used to provide for interaction with a user as well; for example, feedback provided to the user can be any form of sensory feedback (e.g., visual feedback, auditory feedback by way of a microphone 336, or tactile feedback); and input from the user can be received in any form, including acoustic, speech, or tactile input. In the input device 332 and the microphone 336 can be coupled to and convey information via the bus 304 by way of an input device interface 328. Other computing devices, such as dedicated servers, can omit one or more of the display 340 and display interface 314, the input device 332, the microphone 336, and input device interface 328.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

### EXAMPLES

### Example I

### ImmunoGenScore Improved Specificity of Predicting Immunogenic Neoantigen Peptides

The specificity of the NP-predictor and T-epitope predictor scores in predicting neoantigen peptides that are presented by MHC and recognized by T-cell was compared to NetMHCpan. Neoantigen peptides that are generated from cancer cells were used as input for the machine learning models.

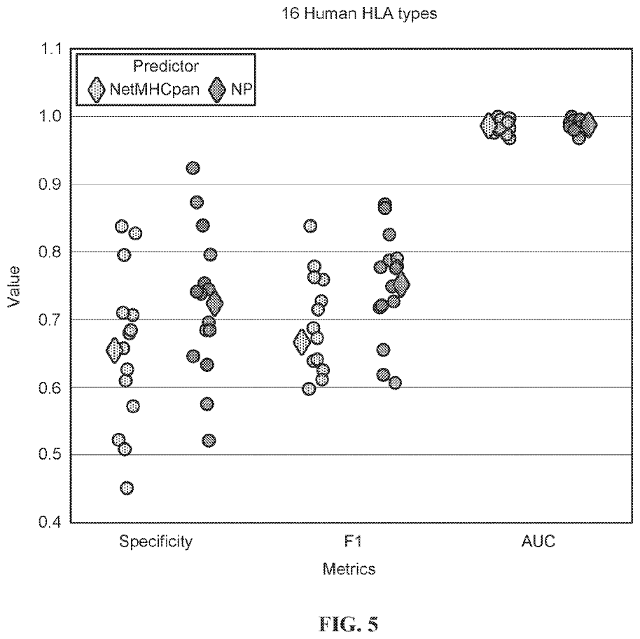

Two independent datasets were used from published studies. For NP-predictor (FIG. 5), the peptides were obtained by high-throughput mass-spectrometry experiments, measuring antigen sequence and abundance eluted after HLA-peptide immunopurification from cells that expressed a single HLA class I (Abelin et al. Immunity, 2017). The dataset included 56,800 positive NP peptides covering 16 HLA types. For each HLA type, a 100× ratio of negative peptides, generated from a random human proteome, were added to the testing data. The HLA-type specific cutoff of predicted NP score was determined by the best F1 score on another independent validation data. Using this cutoff, specificity, sensitivity, and F1 score were calculated for 16 HLA-specific testing data.

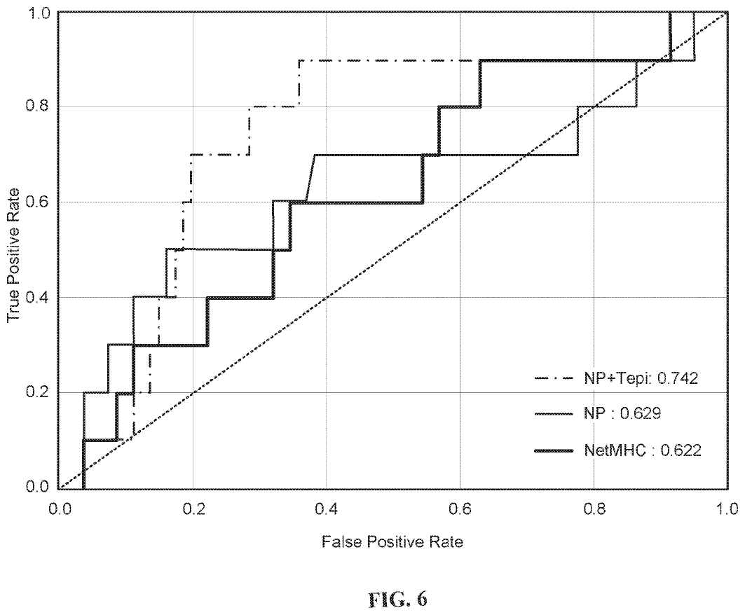

For T-epitope predictor (FIG. 6), the peptides were patient melanoma sample-derived neoantigens, which were also tested for in vitro T-cell reactivity by pulsing autologous CD8+ T-cells and measuring IFN-γ release (Ott et al. Nature, 2017). A total of ten positive and 81 negative 9-mer neoantigens were extracted from the study data and used for testing. The predicted T-epitope scores were compared to true labels (ten “positive” and 81 “negative”) for calculating AUC.

NP-predictor scores and T-epitope predictor scores were generated for each of the neoantigens. Specific HLA alleles were considered for the prediction models. Each of the specific alleles are listed in Table I. In parallel, the neoantigen sequences were subjected to analysis by NetMHC.

The neoantigen testing sequences for NP-predictor and T-epitope predictor were 9-mer peptides. The sequences were directly used as input for machine learning models for each specific HLA type. The same 9-mers were also submitted to NetMHCpan3 software, generating a list of predicted IC50 and log-transformed scores. Specificity was calculated for each NP-predictor using the known testing label and HLA-type specific cutoff, which was determined by the best F1 score using an independent validation dataset. Specificity of NetMHC was also calculated based on the specific HLA-type cutoff, which was determined on the same independent validation dataset. Based on specificity and sensitivity, the F1 score was obtained. The AUC was calculated independent of a cutoff.

The output from the NP predictor score alone showed higher specificity compared to NetMHCpan (FIG. 5). In addition, the combination of specificity with sensitivity (F1 score) was higher on leave-out MHC-eluted, NP peptides detected from HLA mono-allelic cell line compared to NetMHCpan (FIG. 5).

The combination of the NP predictor score and the T-epitope (T-epi) predictor score was also compared to the NetMHC prediction. The results for the true positive rate, as measured by the area under the curve, demonstrated that the true positive rate of the ImmunoGenScore (NP+T-epi) was greater than the true positive rate of the NetMHC score (NP+T-epi area=0.742, vs. NetMHC area=0.622) (FIG. 6). Consistent with the specificity analysis, the NP-predictor alone was also found to display a higher true positive rate relative to the NetMHC prediction (0.629 vs. 0.622, respectively) (FIG. 6). combination of T-epi to the NP-predictor improved the true positive rate (0.742 vs. 0.629) (FIG. 6).

Taken together, the results demonstrated that the ImmunoGenScore (NP-predictor+T-epitope-predictor) show better accuracy on predicting probabilities of MHC-presenting and T-cell-interacting. In particular, a considerable improvement has been observed for the novel T-epitope-predictor with immunogenic neoantigen from actual tumor samples.

### Example II

### ImmunoGenScore Produced a Better True Positive Rate than NetMHC Platforms

The true positive rate for ImmunoGenScore was compared to three different versions of NetMHC: NetMHCpan3, NetMHC3, and NetMHC4. The data used for testing was obtained from the IEDB T-cell epitopes for the mouse MHC allele, which were not included in the training of NP- and T-epitope predictors (www.iedb.org/result_v3.php?cookie_id=60f023). The data included a total of 400 9-mer peptides from a variety of diseases and pathogens with experimentally determined cytotoxicity, cytokine release, IFN-γ release, or TCR binding. The true labels were defined in the IEDB database with either a “Positive” or a “Negative” T-cell assay readout. For NetMHCx predictions, the output binding affinity was log-transformed to a [0,1] scale and used for plotting the Receiving Operating Characteristic curve. For ImmunoGen predictions, the output score was directly used for plotting ROC curve.

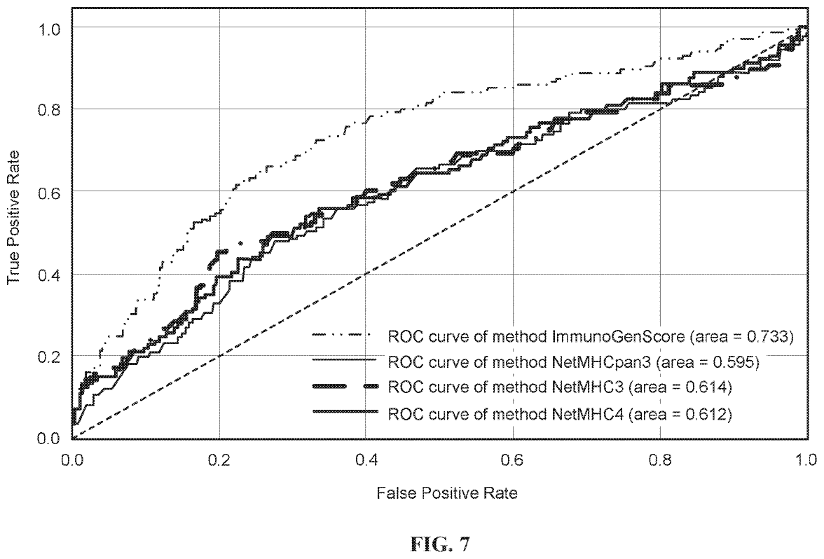

The results demonstrated that the ImmunoGenScore was able to produce a better true positive rate than any of the NetMHC platforms. Specifically, the ImmunoGenScore prediction yielded a ROC curve of 0.733, whereas the NetMHCpan3 had a ROC curve 0.595, the NetMHC3 had a ROC curve of 0.614, and the NetMHC4 had a ROC curve of 0.612 on human melanoma-derived immunogenic neoantigens (FIG. 7). Taken together, these data demonstrate that the ImmunoGenScore is able to give a more accurate prediction of immunogenic neoantigen peptides.

### Example III

### ImmunoGenScore Produced Superior Prediction of Peptides with CD8+ T-Cell Reactivity

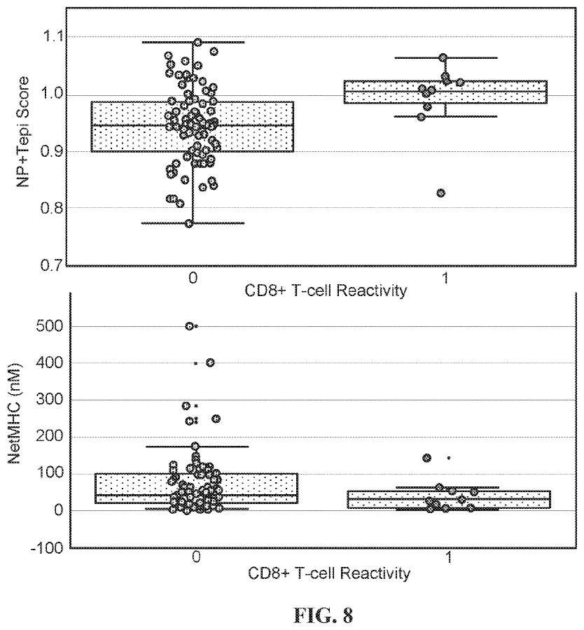

Comparison of the NP-predictor score+T-epitope predictor score (ImmunoGenScore) was compared to the NetMHC affinity prediction for CD8+ T-cell reactivity. Peptides from patient melanoma sample-derived neoantigens were tested for in vitro T-cell reactivity by pulsing autologous CD8+ T-cells and measuring IFN-γ release (Ott et al. Nature, 2017). A total of 10 positive and 81 negative 9-mer neoantigens were extracted from the publication and used for testing. The predicted ImmunoGenScore were analyzed against their true labels (10 “+” and 81 “−”) using a Mann-Whitney test to calculate their p value (FIG. 8). In parallel, these peptides were submitted to NetMHC4 to generate HLA-specific prediction of binding affinities. The affinities were not log-transformed, but directly used for plotting the boxplot and calculating Mann-Whitney p-value (FIG. 8)

The results indicated that the ImmunoGenScore was able to better predict peptides that exhibited CD8+ T-cell reactivity (“1”) vs. non-reactivity (“0”) (FIG. 8). In contrast, the NetMHC prediction exhibited no difference between peptides that exhibited CD8+ T-cell reactivity vs. those that were non-reactive.

### Example IV

### Combined ImmunoGenScore and MHC-Binding Score Yield PCV Candidates

Based on the results from the previous examples, the MHC-binding affinity predictor was integrated into the computational pipeline to score the potential neoantigens epitopes by both ImmunoGenScore and MHC-binding score (FIG. 9). This was performed by first combining three testing datasets (Robbins et al, Nature Medicine 2013; Stronen et al. Science 2016; Ott et al. Nature 2017), which resulted in a total of 31 positive neoantigen epitopes and 275 negative neoantigen epitopes. The predictions were generated with NP-, T-epitope predictors and NetMHCpan. The log-transformed NetMHCp and binding affinities and the ImmunoGenScore were plotted on the x- and the y-axis, respectively. Based on the positive and negative labels, two ROCs were plotted for the ImmunoGenScore and the MHC-binding score. The cut-off points were selected corresponding to the highest True Positive Rate×(1−False Positive Rate) on the ROC curves. The cut-off points were then drawn on the 2-D quadrat to establish an optimal neoantigen immunogenicity threshold. On the combined leave-out testing set, the double-positive group was found to have a true discovery rate of 27.1% for immunogenic tumor neoantigens based on the two cut-off points. In comparison, the true discovery rate was 11.2% when the MHC-binding score was used (FIG. 9, shaded area).

In addition, the orthogonal scoring system allowed 38.9% false positive predictions to be excluded (FIG. 5, lower left corner). These results demonstrate that the NP+T-epi score can be combined with the MHC-binding score to deliver fewer false positives and a higher percentage of true positives for selecting neoantigens as PCV candidates.

TABLE 1
HLA or H-2 alleles considered in NP or T-epitope predictor models

| | | NP predictor | T-epitope predictor |
|---|---|---|---|
| Human Class | HLA-A*01:01 | X | X |
| I HLA | HLA-A*02:01 | X | X |
| | HLA-A*02:02 | | X |
| | HLA-A*02:03 | X | X |
| | HLA-A*02:04 | X | |
| | HLA-A*02:05 | | X |
| | HLA-A*02:06 | | X |
| | HLA-A*02:07 | X | |
| | HLA-A*02:19 | | X |
| | HLA-A*03:01 | X | X |
| | HLA-A*03:02 | | X |
| | HLA-A*11:01 | X | X |
| | HLA-A*23:01 | X | X |
| | HLA-A*24:02 | X | X |
| | HLA-A*25:01 | X | |
| | HLA-A*26:01 | X | X |
| | HLA-A*29:02 | X | X |
| | HLA-A*30:01 | X | X |
| | HLA-A*30:02 | | X |
| | HLA-A*31:01 | X | X |
| Human Class | HLA-A*32:01 | X | X |
| I HLA | HLA-A*33:01 | | X |
| | HLA-A*66:01 | | X |
| | HLA-A*68:01 | | X |
| | HLA-A*68:02 | X | X |
| | HLA-A*80:01 | | X |
| | HLA-B*07:02 | X | X |
| | HLA-B*08:01 | X | X |
| | HLA-B*14:01 | | X |
| | HLA-B*15:01 | X | X |
| | HLA-B*15:02 | | X |
| | HLA-B*15:03 | X | |
| | HLA-B*15:10 | X | |
| | HLA-B*15:11 | X | |
| | HLA-B*15:17 | | X |
| | HLA-B*18:01 | | X |
| | HLA-B*27:05 | X | X |
| | HLA-B*35:01 | X | X |
| | HLA-B*35:03 | | X |
| | HLA-B*37:01 | | X |
| | HLA-B*38:01 | X | |
| | HLA-B*39:01 | X | X |
| Human Class | HLA-B*40:01 | X | X |
| I HLA | HLA-B*40:02 | X | X |
| | HLA-B*42:01 | X | |
| | HLA-B*44:02 | X | X |
| | HLA-B*44:03 | X | X |
| | HLA-B*51:01 | X | X |
| | HLA-B*53:01 | X | X |
| | HLA-B*54:01 | X | X |
| | HLA-B*57:01 | X | X |
| | HLA-B*58:01 | | X |
| | HLA-C*01:02 | X | |
| | HLA-C*04:01 | | X |
| | HLA-C*06:02 | | X |
| | HLA-C*07:01 | X | |
| | HLA-C*12:03 | | X |
| | HLA-C*14:02 | | X |
| Human Class | HLA-DRB*101:01 | X | |
| II HLA | HLA-DRB*103:01 | X | |
| | HLA-DRB*115:01 | X | |
| Mouse | H-2-Db | X | |
| | H-2-Kb | X | |

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A method for identifying an immunogenic neoantigen peptide, the method being implemented by one or more data processors forming part of at least one computing device and comprising:
receiving data characterizing a neoantigen peptide from a subject;
generating, using a first machine learning model and for each neoantigen peptide, a naturally processed (NP) antigen predictor (NP-predictor) score, wherein the first machine learning model is trained using data derived from mass spectrometry of isolated peptides eluted from at least one major histocompatibility complex (MHC) molecule;
generating, using a second machine learning model and for each neoantigen peptide, a T-epitope predictor score, wherein the second machine learning model is trained using experimentally characterized peptides recognized by T-cells;
generating, using a third machine learning model and for each neoantigen peptide, a score predicting affinity of the neoantigen peptide for binding an MHC molecule (MHC binding score); and
generating, for each neoantigen peptide, a composite score based on each of the NP-predictor score, the T-epitope predictor score, and the MHC binding score, wherein the composite score identifies one or more immunogenic neoantigen peptides.

2. The method of claim 1, wherein the neoantigen peptide is identified by:
obtaining a sample from the subject, wherein the sample is derived from a cancer cell;
determining a nucleotide sequence of one or more nucleic acids derived from the sample, wherein determining the nucleotide sequence is optionally performed using at least one of exome, transcriptome, or whole genome sequencing;
translating the nucleotide sequence into an encoded peptide sequence; and
identifying a neoantigen peptide.

3. (canceled)

4. The method of claim 2, further comprising determining the abundance of the neoantigen peptide using exome, transcriptome, or whole genome sequencing.

5. The method of claim 1, wherein the first machine learning model is a neural network.

6. The method of claim 5, wherein the neural network is a convolutional neural network, a recurrent neural network, or a deep learning neural network.

7. The method of claim 1, wherein the MHC molecule is a class I or a class II.

8. The method of claim 57, wherein the second machine learning model is selected from a group consisting of a support vector machine, a Bayesian classifier, a random forest model, a logistic regression model, a boosting classifier model, and a neural network.

9. The method of claim 8, wherein the second machine learning model is independently selected for a specific MHC Class I human leukocyte antigen (HLA) glycoprotein, MHC Class II human HLA, or a mouse H2 glycoprotein, wherein the HLA glycoprotein is optionally a HLA subtype selected from the group consisting of HLA-A, HLA-B, HLA-C, HLA-DRB, HLA-DPA, HLA-DPB, HLA-DQA, HLA-DQB, H2-Db, and H2-Kb, and wherein the HLA subtype is optionally a specific HLA allele to four digits.

10.-11. (canceled)

12. The method of claim 1, wherein the experimentally characterized peptides recognized by T-cells are experimentally characterized by a human T-cell assay, and wherein:
(a) the peptide is optionally a 9-mer antigen;
(b) the T-cell assay optionally measures:
(i) cytokine release;
(ii) cytotoxicity, wherein the cytokine is optionally selected from the group consisting of interferon gamma (IFNγ), tumor necrosis factor alpha (TNFα), interleukin-2 (IL-2), interleukin-4 (IL-4), interleukin-5 (IL-5), interleukin-6 (IL-6), interleukin-8 (IL-8), interleukin-10 (IL-10), interleukin-17 (IL-17), interleukin-21 (IL-21), interleukin-22 (IL-22), granzyme A, and granzyme B; or
(iii) qualitative T-cell binding to an antigen presenting cell (APC), wherein the qualitative T-cell binding is optionally determined by MHC multimer staining;
(c) the T-cell assay is optionally performed ex vivo or in vitro; and/or
(d) the T-cell assay readout is positive or negative.

13.-17. (canceled)

18. The method of claim 1, wherein the experimentally characterized peptides recognized by T-cells are selected from the Immune Epitope Database (IEDB).

19. The method of claim 12, further comprising subjecting the experimentally characterized peptides recognized by T-cells to dimensionality reduction, wherein the dimensionality reduction optionally comprises principal component analysis (PCA), singular value decomposition (SVD), or non-negative matrix factorization (NMF).

20.-21. (canceled)

22. The method of claim 1, further comprising separating a neoantigen peptide longer than nine amino acids into segments of nine amino acids with a same sequence order, wherein the segments of nine amino acid optionally serve as the input for the third machine learning model.

23. (canceled)

24. The method of claim 1, wherein the third machine learning model is a neural network, and wherein the neural network source is optionally NetMHC.

25. (canceled)

26. The method of claim 1, further comprising generating, for each neoantigen peptide, an ImmunoGenScore determined by a summation of the NP-predictor score, and the T-epitope predictor score.

27. The method of claim 1, wherein the composite score is generated, for each neoantigen peptide, by a summation of the NP-predictor score, the T-epitope predictor score, and the MHC binding score.

28. The method of claim 1, wherein the composite score is generated, for each neoantigen peptide, by an orthogonal scoring matrix comprising:
generating, for each neoantigen peptide, an orthogonal rank for the ImmunoGenScore;
generating, for each neoantigen peptide, an orthogonal rank for the MHC binding score;
identifying neoantigen peptides with a high orthogonal rank for the ImmunoGenScore;
identifying neoantigen peptides with a high orthogonal rank for the MHC binding score; and
selecting one or more neoantigen peptides present in either or both of the highest orthogonal rank for the ImmunoGenScore and the highest orthogonal rank for the MHC binding score,
wherein a high orthogonal rank for the ImmunoGenScore is optionally an neoantigen peptide:
(a) in the top 50% of neoantigen peptides; or
(b) in the top 10% of neoantigen peptides.

29.-30. (canceled)

31. The method of claim 1, further comprising prioritizing neoantigen peptides with the highest expression, wherein the expression is optionally determined by RNA-seq.

32. (canceled)

33. The method of claim 1, wherein the immunogenic neoantigen peptide is a target for an anti-cancer vaccine.

34. The method of claim 1, wherein a higher composite score indicates the immunogenic neoantigen peptide is an optimal target for an anti-cancer vaccine, and optionally wherein the personalized cancer vaccine (PCV) score.

35. (canceled)

36. A system comprising:
at least one programmable data processor; and
memory storing instructions which, when executed by the at least one programmable data processor, implement operations comprising:
receiving data characterizing a neoantigen peptide from a subject;
generating, using a first machine learning model and for each neoantigen peptide, a naturally processed (NP) antigen predictor (NP-predictor) score, wherein the first machine learning model is trained using data derived from mass spectrometry of isolated peptides eluted from at least one major histocompatibility complex (MHC) molecule;
generating, using a second machine learning model and for each neoantigen peptide, a T-epitope predictor score, wherein the second machine learning model is trained using experimentally characterized peptides recognized by T-cells;
generating, using a third machine learning model and for each neoantigen peptide, a score predicting affinity of the neoantigen peptide for binding an MHC molecule (MHC binding score); and
generating, for each neoantigen peptide, a composite score based on each of the NP-predictor score, the T-epitope predictor score, and the MHC binding score, wherein the composite score identifies one or more immunogenic neoantigen peptides.

37. (canceled)
